(19) United States
(12) Patent Application Publication
(10) Pub. No.: US 2009/0155827 A1
(43) Pub. Date: Jun. 18, 2009

(54) PIGF and FLT-1 as Prognostic Parameters for Cardiovascular Diseases

(75) Inventors: Andreas M. Zeiher, Frankfurt (DE); Christopher Heeschen, Munich (DE); Stefanie Dimmeler, Frankfurt (DE)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: DADE BEHRING MARBURG GMBH, Marburg (DE)

(21) Appl. No.: 11/666,164

(22) PCT Filed: Oct. 25, 2005
(86) PCT No.: PCT/EP05/11443

§ 371 Date: Nov. 12, 2008

(30) Foreign Application Priority Data
Oct. 25, 2004 (DE) 10 2004 051 847.5

(51) Int. Cl. C12Q 1/48; C12Q 1/00

(52) U.S. Cl. 435/15; 435/4

(57) ABSTRACT

The present invention refers to a use of an ex vivo method comprising the determination of PlGF and sFlt-1 in a sample for diagnosis, risk stratification and/or monitoring of a vascular disease with atherosclerotic etiology, in particular a coronary heart disease such a unstable angina pectoris or myocardial infarction, and/or for estimation of the probability of developing such a disease, as well as for identification of a patient supposed to benefit from a therapy by agents reducing the risk for a cardiovascular disease. In the method (i) a ratio of [PlGF=high:sFlt-1=low], and/or (ii) a PlGF concentration in the upper two tertiles of a reference collective, and an sFlt-1 concentration in the lower tertile of the reference collective, and/or (iii) a PlGF result above a PlGF reference value, and an sFlt-1 result below an sFlt-1-reference value indicate an elevated probability for an adverse event. The present invention also refers to the used method. The present invention further refers to a diagnostic kit and its use as well as to an assay element and its use.

This is a U.S. National Stage Application of International Application No. PCT/EP2005/011443, filed Oct. 25, 2005, and claims priority under 35 U.S.C. § 119 to German Application No. 10 2004 051 847.5, filed Oct. 25, 2004. The complete disclosures of both applications are incorporated herein by reference.

The present invention refers to a use of an ex vivo method comprising the determination of PlGF and Flt-1 in a sample with the purpose of diagnosis, risk stratification and/or monitoring of a vascular disease with atherosclerotic etiology, and/or for estimation of the probability of developing such a disease. The present invention also refers to the used method. The invention further refers to a diagnostic kit and its use.

### BACKGROUND OF THE INVENTION

Inflammatory processes play a fundamental role in all stages of an atherosclerosis, i.e. from the development of early atherosclerotic lesions and their progression to the point of erosion or rather rupture of the lesions associated with the corresponding thrombotic complications. Convincing findings indicate that inflammatory mechanisms are also involved in destabilization of an atherosclerotic lesion resulting in an acute coronary syndrome (1, 2).

Due to the relationship between inflammation and atherosclerosis, established markers of inflammation released into the circulation, are also considered in risk stratification in patients having an acute coronary heart disease. In contrast, for example, to the troponins being markers of cell necrosis, and thus indicating the endpoint of myocardial infarction, markers of inflammation are capable of indicating a respective risk prior to the occurrence of myocardial damage, since they reflect inflammatory processes underlying an acute coronary syndrome.

Among the established markers of inflammation, C-reactive protein (CRP, herein also referred to as highly sensitive CRP, hsCRP) and fibrinogen have attracted the most attention, and the prognostic value of these markers with regard to mortality and ischemic events was clearly demonstrated (22-24). CRP and fibrinogen were shown in retrospective studies to have a known value as prognostic parameters and thus are to be considered as markers, in addition to troponin T value as prognostic parameters and thus are to be considered as markers, in addition to troponin T (14, 25, 26). CRP was shown to be a marker useful for the long term prognosis in coronary heart disease, however, its value as a marker of the acute phase, i.e., in the context of an acute coronary syndrome, is considered contradictory (14, 27).

As a first result of the CAPTURE study, only troponin T allowed reliable predictions in the early phase of 72 hours following the onset of symptoms of an acute coronary syndrome, whereas both troponin T and CRP were independent prognostic parameters of a risk within the subsequent six months (14). Comparable results were reported for the GUSTO IV-ACS study (27). The precise source of the elevated CRP levels in patients having an unstable coronary disease further remains unclear. In connection with the assumption that damage of the myocardium also represents a significant stimulus of inflammation, it must be recognized that in a more recent combined analysis of FRISC-II and GUSTO-IV, an elevation of CRP during a time period of up to 120 hours was found in patients only having elevated levels of troponin (27). Similarly, CRP levels were significantly elevated in troponin-positive patients of the CAPTURE study (14), indicating that an acute inflammatory process based on myocardial damage is overlaying a chronic inflammation in the vessel wall, with the result that the chronic inflammatory process associated with an acute coronary syndrome can hardly be estimated using CRP. Furthermore, it should be noted that proinflammatory cytokines are also released by adipose tissue, tissue macrophages, and injured myocardium.

Only recently, the placental growth factor (PlGF), a member of the vascular endothelial growth factor (VEGF) family, was shown to be expressed at elevated levels in early and advanced atherosclerotic lesions (3). Originally identified in placenta (4), PlGF stimulates vessel smooth muscle cell growth, recruits macrophages into atherosclerotic lesions, promotes the production of various inflammatory mediators in macrophages (tumour necrosis factor-α, TNF-α, monocytic chemotactic protein-1, MCP-1, proteases), and stimulates pathologic angiogenesis in the vessel wall (3, 5). Inhibition of the effects of PlGF by blocking its membrane receptor Flt-1 (Fms-like tyrosine kinase-1) in an animal model of atherosclerosis suppressed the growth of atherosclerotic plaques and showed beneficial effects on their stability by inhibiting macrophage infiltration (3, 6). In patients having acute coronary heart disease, it was recently shown that represents a powerful clinical marker of vascular inflammation, with corresponding adverse implications for the patient (7).

In addition to PlGF, Flt-1 also binds to the related factor VEGF (8) and occurs in two forms: a membrane-bound receptor tyrosine kinase of Flt-1 transducing the angiogenic signals inside the cell, and as a soluble ectodomain (soluble Flt-1, sFlt-1) having the function of scavenging the factors PlGF and/or VEGF circulating in free form (6). Since a cytosolic domain is missing from the soluble form of Flt-1, the function of sFlt-1 is restricted to the regulation of the amount of circulating PlGF or VEGF which are available as free factors for activation of the membrane-bound receptors Flt-1 and Flk-1 (fetal liver kinase-1) (9). During an acute coronary heart syndrome, elevated concentrations of the soluble PlGF receptor sFlt-1 could be detected (10).

Patent application WO 2004/046722 (Dimmeler et al.) discloses a method for the analysis of samples in the context of acute cardiovascular diseases, the method comprising the measurement of concentrations of a marker, e.g. PlGF, and optionally of an additional marker, e.g. VEGF, or another marker of inflammation.

A method for the diagnosis of preeclampsia or eclampsia is known from patent application US 2004/126828 (Karumanchi et al.) comprising the measurement of sFlt-1, VEGF, or PlGF concentration. sFlt-1 has been described as a possible candidate for a factor of preeclampsia (17), since not only the placenta of pregnant women with preeclampsia produces elevated amounts of sFlt-1, but elevated sFlt-1 levels point to later development of preeclampsia (18). In US 2004/126828, an elevated concentration of sFlt-1, in particular serum levels of>2,000 mg/l, and a decreased concentration of VEGF, are regarded as positive diagnostic indicators of preeclampsia. When the results obtained from the three markers are correlated in order to determine the so-called “angiogenic index,” the diagnosis of a manifested preeclampsia or a considerable risk for its development-can be made when the angiogenic index, estimated according to the formula [sFlt-1/VEGF+PlGF], is >20, i.e., whenever the sFlt-1 concentration is at least 20-fold greater that of the sum of the concentrations of VEGF and PlGF.

Patent application WO 2005/031364 (Thadhani and Karumanchi) describes a method for diagnosis or prognosis of a gestosis such as preeclampsia comprising the measurement of sexual hormone binding globulin (SHBG) and PlGF, and in a particular embodiment of sFlt-1.

As seen from patent application WO 2005/017192 (Thadhani et al.) serum levels of PlGF determined in preeclampsia were considerably lower (about 6-fold), and those of sFlt-1 were higher (2-fold) compared to results obtained from the measurement of control samples. Accordingly, the ratio of sFlt-1 and PlGF in preeclampsia is 15-fold higher than that of the factor determined in a control sample.

Patent Application WO 98/28006 discloses a method for the diagnosis of hypertension in pregnancy (preeclampsia) by estimating, in a sample, the amount of PlGF, VEGF, and a soluble VEGF receptor such as sFlt-1.

The clinical picture of preeclampsia and eclampsia, respectively, however, is based on a completely different etiology compared to that of a coronary heart disease. In particular, these do not result from an atherosclerotic disease. Therefore, the methods disclosed in the prior art are not transferable to vascular diseases with atherosclerotic etiology, as represented by a coronary heart disease.

Starting from the prior art, it was therefore an object of the present invention to provide a method allowing for an estimation of the probability of developing, for diagnosing, for stratifying risk, and/or for monitoring vascular disease having an atherosclerotic etiology, on the basis of a measurement of biomarkers.

### SUMMARY OF THE INVENTION

It is among the objects of the present invention to provide the methods, uses, and means according to the invention and as defined in the claims.

It is also among the objects of the present invention to provide a method for diagnosis, risk stratification and/or monitoring of a vascular disease with atherosclerotic etiology, and/or for estimation of the probability of developing such a disease, the method comprising the following steps:
(a) providing a patient sample for analysis;
(b) quantifying the PlGF in said sample; and
(c) quantifying the sFlt-1 in said sample.
   Optionally, the method can also comprise the following step:
(d) determining a ratio of the PlGF quantified in (b) and the sFlt-1 quantified in (c).

“Determining a ratio of the PlGF quantified in (b) and the sFlt-1 quantified in (c)” comprises the calculation of the quotient of the “PlGF quantified in (b)/quantified sFlt-1 quantified in (c),” as well as other alternatives for relating the PlGF quantified in (b) to the sFlt-1 quantified in (c).

It is also among the objects of the present invention to provide a method for diagnosis, risk stratification and/or monitoring of a vascular disease with atherosclerotic etiology, and/or for estimation of the probability of developing such disease, comprising the following steps:
(a) providing a patient sample for analysis;
(b) quantifying the PlGF in said sample;
(c) quantifying the sFlt-1 in said sample; and
(d) comparing each of the results of PlGF and sFlt-1 obtained in (b) and (c) to a reference value and/or to a result obtained in a reference sample.

Optionally, the method comprises the following steps:
(a) providing a patient sample for analysis;
(b) quantifying the PlGF in said sample;
(c) quantifying the sFlt-1 in said sample;
(d′) determining a ratio of the PlGF quantified in (b) and the sFlt-1 quantified in (c), preferably calculating the quotient of PlGF/sFlt-1 and/or the quotient of sFlt-1/PlGF; and
(e′) comparing the result obtained in (d′) to a reference value and/or to a result obtained in a reference sample.

Optionally, the method comprises the following steps:
(a) providing a patient sample for analysis;
(b) quantifying the PlGF in said sample;
(c) quantifying the sFlt-1 in said sample;
(d) comparing each of the results of PlGF and sFlt-1 obtained in (b) and (c) to a reference value and/or to a result obtained in a reference sample;
(d′) determining a ratio of the PlGF quantified in (b) and the sFlt-1 quantified in (c), preferably calculating the quotient of PlGF/sFlt-1 and/or the quotient of sFlt-1/PlGF; and
(e′) comparing the result obtained in (d′) to a reference value and/or to a result obtained in a reference sample.

Steps (b) and (c) can be carried out sequentially in the above order, in reverse order, or at the same time.

In step (d), the result obtained in (b) is compared to a reference value of PlGF and/or the amount of PlGF determined in a reference sample, and the result obtained in (c) is compared to a reference value of sFlt-1 and/or the amount of sFlt-1 determined in a reference sample. In step (e′), the result obtained in (d′) (in particular the quotient of PlGF/sFlt-1 and/or the quotient of sFlt-1/PlGF) is compared to a reference value for the relationship of PlGF and sFlt-1 and/or to a result referring to this relationship, determined in a reference sample.

The present invention is a method carried out ex vivo, i.e. an in vitro method.

The wording “vascular disease with atherosclerotic etiology” excludes the diseases of pre-eclampsia and eclampsia, respectively. The term “atherosclerotic” refers both to stable and unstable atherosclerosis.

The term “providing” of a sample to be analyzed, as used herein, is to be equated with “making available.” This means that an available sample to be analyzed is subjected to in vitro measurement, for example by introduction into a measuring instrument. The sample to be analyzed, preferably blood plasma or serum, and/or the reference sample, can be pre-treated, for example by addition of an anti-coagulant to peripheral blood, particularly EDTA, heparin, or citrate. The term “providing” does not comprise the sample collection per se, for example the invasive withdrawal of a sample of a patient such as by puncture, or a non-invasive sample collection such as collection of a sample of urine.

In a preferred embodiment of the present invention the patient is a mammal, particularly preferably a human. The term “patient” particularly refers to a person treated by a medical doctor or other medical staff and comprises sick or ill individuals as well as healthy individuals or apparently healthy individuals.

“Quantifying” PlGF and/or sFlt-1 can be performed by determining a concentration, for example a protein concentration. In addition to determining a concentration, e.g., in blood plasma or serum, quantifying can be performed by determining the amount of molecules, e.g., in a histologic tissue section. “Quantifying” also comprises semiquantitative methods of detection which measure only approximate amounts or concentrations of PlGF and/or sFlt-1 in a sample or serve only for a relative indication of an amount or concentration or inform only as to whether the amount or concentration of PlGF and/or sFlt-1 in the sample is below or above a particular reference value, or more than one particular reference value.

The term “reference value” can be a predetermined value or a value determined in the reference sample. A “reference sample” can be derived, for example, from healthy individuals or from patients having or not having a stable or unstable atherosclerosis, preferably from patients having an acute coronary syndrome, particularly preferably from patients having unstable angina pectoris or an acute myocardial infarction. A sample to which PlGF and sFlt-1 have been added in a ratio measured earlier in healthy individuals or in patients having a vascular disease with atherosclerotic etiology can also be considered. Usually, different reference samples were employed indicating the various possible prognoses, for example “adverse event not probable” to the point of “adverse event highly probable.” The provision of reference samples is preferably made in the same manner as the provision of the sample to be analyzed. In place of the application of reference samples, predetermined reference values to be read, for example, from a table, can also be used. Such reference values can, for example, predetermine different ranges indicating the probability of an event.

Preferably, a reference value and/or a value detected in a reference sample is a “cut-off value” or a “threshold value” or a “critical value,” i.e., a value indicating a limit or threshold. A comparison of a result measured in a sample to a cut-off value shows that a result above the limit or threshold leads to an assessment other than a result below the limit or threshold. In the present invention, for example, a PlGF concentration would be regarded as a suitable PlGF cut-off value which divides the two upper tertiles of an appropriate reference collective from the lower tertile. Another appropriate PlGF cut-off value is that PlGF concentration which separates the upper tertile of an appropriate reference collective from the median tertile. An appropriate sFlt-1 cut-off value, for example, is that sFlt-1 concentration which separates the median tertile of an appropriate reference collective from the lower tertile. In addition to the respective cut-off value determined by tertiles, cut-off values suitable in the present invention can be determined also by means of receiver operating curves (ROC) and other established methods (see also “A. PATIENTS AND METHODS, 4. Statistical methods”). Thus, the median PlGF or sFlt-1 concentration determined using an appropriate reference collective can serve as a PlGF cut-off and sFlt-1 cut-off value, respectively. In the present invention, exceeding an appropriate PlGF cut-off value while simultaneously falling below an appropriate sFlt-1 cut-off value would indicate an increased probability for the respective patient of experiencing a myocardial infarction or stroke and/or of dying from a vascular disease with atherosclerotic etiology.

A particularly favourable method according to the invention is the use of a method comprising the following steps.
(a) providing a patient sample for analysis;
(b) quantifying the PlGF in said sample;
(c) quantifying the sFlt-1 in said sample; and
(d) comparing each of the results of PlGF and sFlt-1 obtained in (b) and (c) to a reference value and/or to a result obtained in a reference sample,
   for diagnosis, risk stratification and/or monitoring of a vascular disease with atherosclerotic etiology in a patient, and/or for estimation of the probability for a patient of developing such a disease.

Optionally, the use of the method comprises the following steps:
(a) providing a patient sample for analysis;
(b) quantifying the PlGF in a sample;
(c) quantifying the sFlt-1 in a sample;
(d′) determining a ratio of the PlGF quantified in (b) and the sFlt-1 quantified in (c), preferably calculating the quotient of PlGF/sFlt-1 and/or the quotient of sFlt-1/PlGF; and

(e′) comparing the result obtained in (d′) to a reference value and/or a result obtained in a reference sample.

Optionally, the use of the method comprises the following steps:
(a) providing a patient sample for analysis;
(b) quantifying the PlGF in said sample;
(c) quantifying the sFlt-1 in said sample;
(d) comparing each of the results of PlGF and sFlt-1 obtained in (b) and (c) to a reference value and/or to a result obtained in a reference sample;
(d′) determining a ratio of the PlGF quantified in (b) and the sFlt-1 quantified in (c), preferably calculating the quotient of PlGF/sFlt-1 and/or the quotient of sFlt-1/PlGF; and
(e′) comparing the result obtained in (d′) to a reference value and/or a result obtained in a reference sample.

Steps (b) and (c) can be carried out sequentially in the above order, in reverse order, or at the same time.

It is among the objects of the invention to provide a diagnostic kit comprising at least one means for quantifying PlGF and at least one means for quantifying sFlt-1 in a sample to be analyzed, wherein the kit can also consist of separate packages, and wherein the kit further comprises an information means (e.g., a package insert), according to which (i) a ratio of [PlGF=high:sFlt-1=low] and/or (ii) a PlGF concentration in the two upper tertiles of a reference collective, and an sFlt-1 concentration in the lower tertile of the reference collective, and/or (iii) a PlGF result above the PlGF reference value and an sFlt-1 result above the sFlt-1 reference value indicates, for example, an elevated probability of an adverse event such as death, non-fatal myocardial infarction, and/or stroke.

It is among the objects of the present invention to provide a use of the kit according to the invention for diagnosis, risk stratification, and/or monitoring of a vascular disease with atherosclerotic etiology, and/or for estimation of the probability of developing such a disease.

It is among the objects of the present invention to provide a use of the kit according to the invention for carrying out the method according to the invention.

In the following, further details and explanations shall be added to the description of the invention.

In one embodiment of the method and of the use of the method, the vascular disease is selected from the group consisting of an organ related vascular disease (in particular a coronary heart disease or a cerebrovascular disease) and/or a peripheral vascular disease (in particular an arterial or venous occlusive disease). In a further embodiment of the method, the vascular disease is a coronary syndrome, preferably unstable angina pectoris or acute myocardial infarction. In a preferred embodiment of the method, the coronary heart disease is an acute coronary syndrome. In a preferred embodiment of the method, samples are used only of patients suffering from a vascular disease as more detailed above, in particular of an acute coronary syndrome (e.g., a myocardial infarction), or who are suspected to have such a disease or to develop such a disease in the future. The sample can also be derived from “randomly” selected patients, for example in the context of a screening or a preventive medical check-up. Preferably, the method according to the invention is used in an acute coronary syndrome, such as angina pectoris and/or acute myocardial infarction.

The sample to be analyzed preferably is peripheral blood or a fraction thereof, particularly preferred is the fraction of blood plasma (plasma) or blood serum (serum). In another embodiment of the invention also other bodily fluids (e.g., urine or liquor) and tissue specimens, suspensions of tissue cells, tissue homogenates or tissue sections are used as samples to be analyzed. A “sample” for the purpose of the invention is a material supposed to contain PlGF and sFlt-1 as detectable substances. Where appropriate, the samples must be pretreated in order to render the substances to be detected available for the respective analytical procedure or in order to remove interfering components from the sample. Such a pre-treatment of samples may include the separation and/or lysis of cells, precipitation, hydrolysis or denaturation of sample components such as proteins, centrifugation of samples, treatment of the sample with organic solvents such as alcohols, in particular methanol, or treatment of the sample with detergents.

In a preferred embodiment of the present invention, the patient is a mammal, preferably a human, and particularly preferably a human having a vascular disease, as further detailed above, preferably having an acute coronary syndrome, such as a myocardial infarction. In a particularly preferred embodiment of the method according to the invention, samples of patients are analyzed only if pregnancy can be excluded or can be excluded at least with the utmost probability.

In a preferred embodiment, the method according to the invention is used for risk stratification of a vascular disease with atherosclerotic etiology or the method comprises carrying out a risk stratification. Risk stratification comprises the determination of a probability for a patient of experiencing an adverse event such as death, non-fatal myocardial infarction, and stroke. The adverse event can also be an adverse after-effect consisting of, for example, experiencing a further non-fatal myocardial infarction, experiencing stroke after a first non-fatal myocardial infarction, or death.

The methods according to the invention indicate an elevated probability for an adverse event (i) at a PlGF value above the PlGF reference value and an sFlt-1 value below the sFlt-1 reference value, and/or (ii) at a PlGF concentration in the two upper tertiles of a reference collective and an sFlt-1 concentration in the lower tertile of the reference collective, and/or (iii) at a ratio of [PlGF=high:sFlt-1=low].

The term “reference collective” normally refers to a group of reference individuals, preferably randomly selected from the entirety of a population meeting certain selection criteria. For practical reasons, a reference collective is often established on the basis of practical considerations, i.e., appropriate individuals being simply available are selected, instead of randomly selecting individuals from an entirety of a population or an overall collective. Most clearly defined selection criteria are, for example, defined and typical diseases, for example unstable angina pectoris, acute myocardial infarction, etc. Additionally, reference collectives of healthy individuals, undifferentiated and hospitalized individuals, etc., are relevant in order to determine population based reference values for the respective collectives. The reference collective preferred with regard to the present invention consists of a number of individuals suffering from a vascular disease with atherosclerotic etiology, in particular from an acute coronary syndrome such as unstable angina pectoris or acute myocardial infarction, the number of individuals being sufficient for statistical purposes. Reference collectives can also be recruited from patients showing an elevated or decreased incidence of events.

In addition to reference values based on a reference collective, “subject-based reference values” can also be employed. Subject-based reference values are values already available (e.g., a concentration of a biomarker such as PlGF or sFlt-1 of one single individual determined at a time when the individual was in a defined state of health or disease).

In a preferred embodiment of the method according to the invention, a PlGF cut-off value of ≧17.7 ng/l is used as a reference value. In a further preferred embodiment of the method according to the invention, a PlGF cut-off value of ≧23.3 ng/l is used as a reference value. A PlGF cut-off value of ≧15.6 ng/l can be used as well. A PlGF cut-off value in the range of 15.6 to 23.3 ng/l is preferably used, particularly preferably in the range of 10 to 50 ng/l, more particularly preferably in the range of 5 to 100 ng/l, and even more particularly preferably in the range of 1 to 500 ng/l.

In a preferred embodiment of the method according to the invention, an sFlt-1 cut-off value of ≦37.4 ng/l is used as a reference value. In another preferred embodiment of the method according to the invention, an sFlt-1 cut-off value of ≦56.5 ng/l is used as a reference value. An sFlt-1 cut-off value in the range of 37.4 to 56.5 ng/l is preferably used, particularly preferably in the range of 25 to 100 ng/l, more particularly preferably in the range of 10 to 250 ng/l, and even more particularly preferably in the range of 5 to 500 ng/l.

In a particularly preferred embodiment of the method according to the invention, a concentration of PlGF of >17.7 ng/l refers to a high and a concentration of PlGF of <17.7 ng/l refers to a low PlGF concentration. In an alternative, particularly preferred, embodiment of the method according to the invention, a concentration of PlGF of >23.3 ng/l refers to a high, of 15.6 to 23.3 ng/l refers to a medium, and of <15.6 ng/l refers to a low PlGF concentration.

In a particularly preferred embodiment of the method according to the invention, a concentration of sFlt-1 of >56.5 ng/l refers to a high and a concentration of PlGF of <56.6 ng/l refers to a low sFlt-1 concentration. In an alternative, particularly preferred, embodiment of the method according to the invention, a concentration of sFlt-1 of >91.4 ng/l refers to a high, of 37.4 to 91.4 ng/l refers to a medium, and of <37.4 ng/l refers to low sFlt-1 concentration.

The determination of a “ratio” of PlGF and sFlt-1 can be done by calculating a quotient of PlGF/sFlt-1. Alternatively, a quotient of sFlt-1/PlGF can be determined as well. A quotient of ≧0.31, based on a ratio of [PlGF>17.7 ng/l: sFlt-<56.6 ng/l], preferably indicates an elevated risk for an adverse event. A quotient of ≧0.42, [PlGF>15.6 ng/l: sFlt-1<37.4 ng/l] is particularly preferred as an indicator of an elevated risk for an adverse event. A quotient of ≧0.62 [PlGF>23.3 ng/l: sFlt-1<37.4 ng/l] is more particularly preferred as an indicator of an elevated risk for an adverse event. The determination of a ratio can also mean to correlate, for example by simple comparison, the results of PlGF and sFlt-1.

In one embodiment, the method according to the invention comprises quantifying at least one additional biomarker. In a preferred embodiment, the additional biomarker is selected from the group consisting of VEGF, sCD40L, PAPP-A (pregnancy associated plasma protein-A), MPO (myeloperoxidase), cystatin C, myoglobin, creatine kinase, in particular creatine kinase MB (CK-MB), troponin, in particular troponin I, troponin T and/or its complexes, CRP, natriuretic peptides such as ANP (atrial natriuretic peptide), BNP (B-type natriuretic peptide) or NT-proBNP. Further biomarkers are also hematopoietins such as EPO (erythropoietin), GM-CSF (granulocyte/macrophage colony-stimulating factor), G-CSF (granulocyte colony-stimulating factor), LIF (leukemia inhibition factor), oncostatin, CNTF (ciliary neurotrophic factor), myoglobin, Lp-PLA₂ (lipoprotein associated phospholipase A₂), IMA (ischemia modified albumin), cysteinylated albumin, GP-BB (glycogen phosphorylase isoenzyme BB), H-FABP (heart-type fatty-acid-binding protein), choline, PPARs (peroxisome proliferator activator receptors), ADMA (asymmetric dimethylarginine), SAA (serum amyloid A protein), fibrinogen, FFAs (unbound free fatty acids), D-dimer, homocysteine, PAI-1 (plasminogen activator inhibitor 1), P-selectin, soluble E-selectin, hemoglobin A1c, urodilatin, thromboxanes (e.g. thromboxane A₂ and 11-dehydro-thromboxane B₂), mitochondrial adenylate kinase isozymes, proMBP (eosinophil major basic protein), OPG (osteoprotegerin), leptin, adiponectin, FSAP (factor seven-activating protease; in particular its so-called Marburg I-mutant), IL-6 (interleukin-6), MIF (macrophage migration inhibition factor), CALCR (calcitonin receptor), glycophorin (in particular truncated glycophorin), growth hormone, prolactin and interleukins, chemokines such as platelet factor 4, PBP (platelet basic protein), MIP (macrophage inflammatory protein), interferons, TNF (tumor necrosis factor), adhesion molecules such as ICAM (intracellular adhesion molecule) or VCAM (vascular adhesion molecule), cytokines, and other growth factors such as FGF (fibroblast growth factor). The term “biomarker” refers to endogenous substances, e.g., proteins, indicating, for example, the occurrence of a pathophysiologic event in an organism.

In one embodiment, the monitoring of a vascular disease with atherosclerotic etiology means the monitoring of a patient being treated with one or more therapeutic agents reducing the risk for a vascular, preferably a cardiovascular disorder.

In another embodiment, the method according to the invention is used for identification of a patient intended to benefit from the treatment by one or more therapeutic agents reducing the risk of a vascular, preferably a cardiovascular disorder. The “benefit” can be a reduction of the risk of experiencing an adverse event such as death, non-fatal myocardial infarction, or stroke. Furthermore, the benefit can be optimized by an individual treatment through specific selection of high risk patients.

Agents reducing the risk of a vascular, preferably a cardiovascular disorder, comprise those selected from the group consisting of sFlt-1, anti-inflammatory agents, anti-thrombotics, anti-platelet agents, fibrinolytics, lipid lowering agents, direct thrombin inhibitors, and glycoprotein IIb/IIIa receptor inhibitors. In a preferred embodiment, the agent is sFlt-1 or is derived from sFlt-1. This can be, for example, a recombinantly produced sFlt-1, a fragment thereof, or derivative thereof.

Anti-inflammatory agents include alclofenac, alclometasone dipropionate, algestone acetonide, alpha-amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelaine, broperamol, budesonide, carprofen, cicloprofen, cintazone, cliprofen, clobetasol propionate, clobetason butyrate, clopirac, cloticasone propionate, cormethason acetate, cortodoxone, deflazacort, desonide, desoximetasone, dexamethasone diisopropionate, diclofenac potassium, diclofenac sodium, diflorasone diacetate, diflumidone sodium, diflunisal, difluprednat, diftalon, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixine, flunixine-meglumine, fluocortin butyl, fluorometholone acetate, fluquazone, flurbiprofen, fluretofen, fluticasone propionate, furaprofen, furobufen, halcinonide, halobetasole propionate, halopredone acetate, ibufenac-ibuprofen, ibuprofen aluminum, ibuprofen-piconol, ilonidap, indomethacin, indomethacin sodium, indoprofen, indoxol, intrazole, isoflupredon acetate, isoxepac, isoxicam, ketoprofen, lofemizole hydrochloride, lornoxicam, loteprednol etabonate, meclofenamate sodium, meclofenamic acid, meclorison dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxine, oxaprozine, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazat, prifelone, prodolic acid, proquazone, proxazole, proxazole citrate, rimexolone, romazarit, salcolex, salnacedin, salsalate, salicylates, sanguinarium chloride, seclazone, sermetacine, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, glucocorticoids, and zomepirac sodium.

Anti-thrombotic and/or fibrinolytic agents include plasminogen (its conversion into plasmin is mediated by prekallikrein, kininogens, factor XII, factor XIIIa, plasminogen proactivator and tissue plasminogen activator [TPA]), streptokinase, urokinase, anisoylated plasminogen-streptokinase activator complex, pro-urokinase (pro-UK), rTPA (alteplase or activase; r=recombinant), rpro-UK, abbokinase, eminase, streptase anagrelide hydrochloride, bivalirudin, dalteparin sodium, danaparoid sodium, dazoxiben hydrochloride, efegatran sulfate, enoxaparin sulfate, ifetroban, ifetroban sodium, tinzaparin sodium, retaplase, trifenagrel, warfarin, and dextrans.

Anti-platelet agents include clopidogrel, sulfinpyrazone, aspirin, dipyridamole, clofibrate, pyridinole carbamate, PGE, glucagon, antiserotonin agent, caffeine, theophylline pentoxifyllin, ticlopidine, and anagrelide.

Lipid lowering agents include gemfibrozil, cholystyramine, colestipole, nicotinic acid, probucol lovastatin, fluvastatin, simvastatin, atorvastatin, pravastatin, and cirivastatin.

Direct thrombin inhibitors include hirudin, hirugen, hirulog, agatroban, PPACK, and thrombin aptamers.

Glycoprotein IIb/IIIa receptor inhibitors both are antibodies and non-antibodies and include ReoPro® (abciximab), lamifiban, and tirofiban, without being restricted to the aforementioned inhibitors.

PlGF and/or sFlt-1 can be detected by immunologic methods, e.g., ELISA, also including a detection of fragments of PlGF and/or sFlt-1, e.g., peptides, and of PlGF and/of sFlt-1 isoforms and derivatives. Alternatively, also the mRNA of PlGF and/or sFlt-1 can be detected. In addition to the above-mentioned ELISA also other immunochemical methods for quantifying PlGF and/or sFlt-1 can be used according to the invention. Heterogenous or homogenous sandwich-immunoassays are particularly suitable, but competitive immunoassays can be used for quantification as well. Usually, monoclonal and polyclonal antibodies as used as specific binding partners in such assays, but instead of antibodies other substances (e.g. heptamers) capable of specifically binding PlGF or sFlt-1 can be employed as well. The term “antibody” does not only refer to complete antibodies, but also explicitly refers to parts, derivatives or homologs of antibodies such as antibody fragments, e.g., Fab, Fv, F(ab′)₂, Fab′, chimeric, humanized, bi- or oligospecific, and single chain antibodies; furthermore, aggregates, polymers and conjugates of immunogluobulins.

The antibodies used in the immunoassays or other specific PlGF or sFlt-1 binding partners can be bound to a carrier consisting of a porous and/or non-porous, generally water-insoluble material, and the carrier can have very varying forms. The carrier can be part of a device such as a vessel, a tube, a microtiter plate, a sphere, a microparticle, a rod, or a strip, as well as filter or chromatography paper.

The antibodies or other specific PlGF or sFlt-1 binding partners can be bound to a detection means (label) generating a signal by itself or inducing the generation of a signal such as a fluorescent substance, a radioactive substance, an enzyme, a microparticle (e.g. an unstained, stained, or otherwise labeled latex particle, a gold sol particle etc.), or a chemiluminescent substance, or the detection means can serve as a mediator (e.g., biotin label) in a detection system (e.g., avidin-peroxidase complex).

The use of assays allowing the quantification of PlGF and sFlt-1 in one test sample is of particular advantage for the purpose of the invention. This can be done, for example, by adding to the sample specific PlGF and sFlt-1 binding partners being bound to different detection means (e.g., to a substance fluorescing at different wavelengths) so that the resulting measuring signals can be measured separately after the immunochemical reaction has been terminated. A particularly advantageous embodiment of such an assay is based on the spatially separated measurement of the measuring signals correlating with PlGF and sFlt-1 concentration, for example, by means of an immunochromatographic assay element as used in principle for the detection of drugs or pregnancy hormones.

In one embodiment of the method according to the invention, the sample and, unless already present in the assay element preferably in dried form, the labeled, Le. associated with a detection means, anti-PlGF antibodies, and anti-sFlt-1 antibodies are applied to the sample application zone of the assay element for quantifying PlGF and sFlt-1. Particularly suitable labels are, for example, stained latex particles, colloidal gold, enzymes, fluorescing substances, radioactive substances or chemiluminescing substances. Provided that PlGF and/or sFlt-1 are contained in the sample, PlGF/antibody complexes and/or sFlt-1/antibody complexes will be formed. These complexes and unbound PlGF or sFlt-1 molecules possibly still present move, e.g., by means of capillary forces, towards the region (detection zone) of the assay element where spatially separated other anti-PlGF antibodies and other anti-sFlt-1 antibodies are fixed, for example, in the form of two bands, or become fixed in the course of the assay procedure (e.g., via a biotin-avidin bridge). Provided that PlGF and/or sFlt-1 are present in the sample, labeled PlGF/antibody sandwich complexes and/or labeled sFlt-1/antibody sandwich complexes will be formed within this detection zone. Unbound components are transported by the stream of fluid to other regions of the assay element. The intensity of the respective signals within the detection zone correlates proportionally to the PlGF and sFlt-1 sample concentration, respectively. Although the above-described sandwich immunoassay procedure is particularly preferred, a competitive assay for quantifying PlGF and sFlt-1 on the basis of such assay elements is possible as well. Instead of one or more antibodies, other substances capable of specifically binding to PlGF or sFlt-1 can also be used, as described above.

A further subject of the present invention therefore is an assay element, for example an immunochromatic assay element, comprising a sample application zone, which may be, for example, a filter paper or another chromatographic means, to which the sample and, unless already being present in the assay element preferably in dried form, the labeled anti-PlGF antibodies and anti-sFlt-1 antibodies can be applied, and wherein the sample application zone is contacting a detection zone with the consequence that a fluid applied to a sample application zone can arrive at the detection zone, e.g. by capillary forces, and wherein the detection zone comprises spatially separated regions for specific binding of PlGF and sFlt-1 with the result that PlGF and sFlt-1 molecules possibly present in the fluid can be bound. Furthermore, the assay element can also comprise an absorption zone, preferably made from highly absorbing material (e.g. filter paper) contacting the detection zone, into which absorption zone unbound components of the stream of fluid are transported. In a further embodiment, this assay element according to the invention additionally comprises means allowing or facilitating the correlation of the signal strength to the PlGF and sFlt-1 sample concentration, respectively, in particular within the clinically relevant range (preferable within the cut-off range). In an alternative embodiment of this assay element according to the invention, use is made of a competitive immunoassay in place of the sandwich immunoassay. In one embodiment of the invention, the assay element is used for carrying out the method according to the invention. In another embodiment of the invention, the assay element is used for diagnosis, risk stratification and/or monitoring of a vascular disease with atherosclerotic etiology in a patient, and/or for estimation of the probability of a patient developing such a disease. Instead of one or more antibodies, other substances specifically binding to PlGF or sFlt-1 can also be employed in this assay element, as described above.

The assay element, which may be a test strip assembled from one or more elements, can have a sample application zone and a detection zone for the detection of each of PlGF and sFlt-1. In one embodiment, the assay element is made of two parallel test strips which may be each assembled from several elements and/or which may be in contact at the sample application zone or at the absorption zone. In one embodiment, two independent assay elements are provided, i.e., one for PlGF and one for sFlt-1. The assay elements can be part of a kit. In a further embodiment, the assay element is used for the method according to the invention.

Since the concentration of a substance, when determined immunochemically, depends on the assay methods used, and in particular on standards and antibodies used, concentrations of a substance determined in two assays with one and the same sample can differ. Provided that, according to the invention, an assay for quantifying PlGF or sFlt-1 is used that differs from that provided in the examples, it is recommended either to convert the concentrations considering a conversion factor or to determine the reference values and tertiles for the assay on the basis of an appropriate reference collective (see e.g., below “A. PATIENTS AND METHODS, A. Patients”), and then to use these results according to the invention. An alignment of the standards between the assays is possible as well.

A subject of the invention is also a reference sample having a PlGF and/or sFlt-1 concentration in the respective cut-off range (particularly as indicated below) for use in the method according to the invention. A preferred reference sample has a PlGF concentration of >15.6 ng/l, preferably of >17.7 ng/l, particularly preferably of >23.3 ng/l, and/or an sFlt-1 concentration of <56.5 ng/l, preferably of <37.4 ng/l. Also preferred is a reference sample having a PlGF concentration in the range of 15.6 to 23.3 ng/l, particularly preferably in the range of 10 to 50 ng/l, most particularly preferably in the range of 5 to 100 ng/l, and even more preferably in the range of 1 to 500 ng/l. Further preferred is also a reference sample having an sFlt-1 concentration in the range of 37.4 to 56.5 ng/l, particularly preferably in the range of 25 to 100 ng/l, most particularly preferably in the range of 10 to 250 ng/l, and even more preferably in the range of 5 to 500 ng/l. A further preferred reference sample has a PlGF concentration in the range of the cut-off value experimentally determined or, for example, a PlGF cut-off value ±25%, particularly preferably ±50%, and most particularly preferably ±100%, as indicated according to the manufacturer's information. A further preferred reference sample has an sFlt-1 concentration in the range of a cut-off value experimentally determined or, for example, an sFlt-1 cut-off value ±25%, particularly preferably ±50%, and most particularly preferably ±100%, as indicated according to the manufacturer's information. The reference sample can also contain agents for stabilization of PlGF and/or s-Flt-1, preferably protease inhibitors. In one embodiment of the invention, the reference sample according to the invention is used in a method for diagnosis, risk stratification and/or monitoring of a vascular disease with atherosclerotic etiology, and/or for estimation of the probability of developing such disease.

In one embodiment, the kit according to the invention comprises at least one means for quantifying PlGF and at least one means for quantifying s-Flt-1 in a sample to be analyzed, optionally consisting of separate packaging units, the kit further comprising at least one reference sample according to the invention. The reference sample can contain (i) PlGF, (ii) sFlt-1 or (iii) PlGF and s-Flt-1. The kit can also comprise the above-described information means. A kit can also comprise one or more assay elements.

A diagnostic kit can comprise additional components and/or auxiliary additives. For example, the kit can contain further explanations on the interpretation of the results of the assays and, if applicable, suggestions for therapy. The kit can also contain one or more assay elements or can consist of one or more assay elements.

### DETAILED DESCRIPTION OF THE INVENTION

The present invention shall be further explained by the following, on the basis of the examples, which make reference to the accompanying figures, the invention not being limited by the examples or the figures. In the figures:

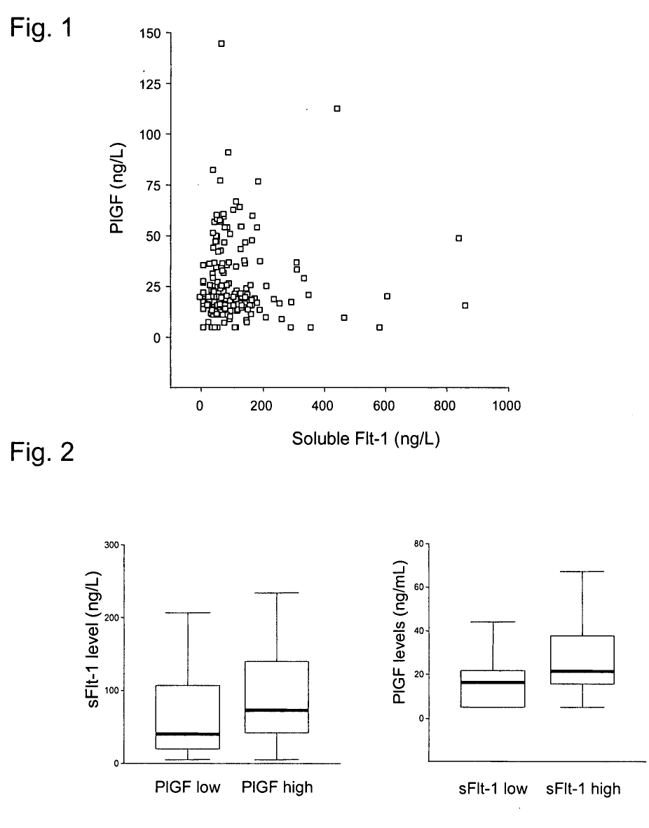

FIG. 1 shows the relationship between the plasma concentrations of sFlt-1 and PlGF.

FIG. 2 shows sFlt-1-concentrations relating to the PlGF initial status, and PlGF concentrations relating to the initial concentration of sFlt-1.

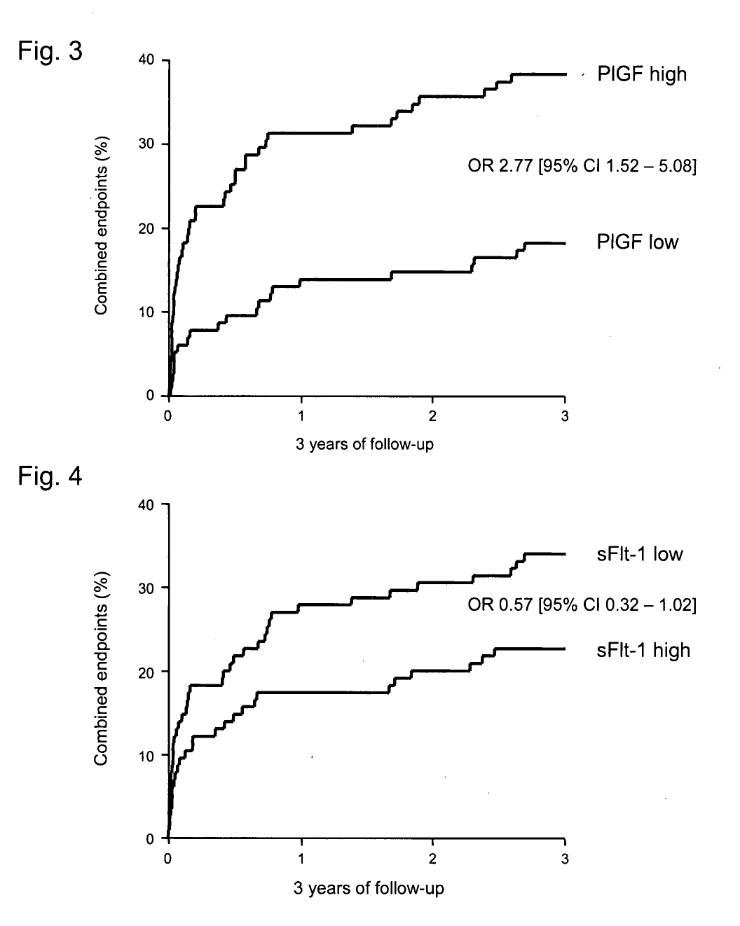

FIG. 3 shows event-rates, calculated according to Kaplan-Meier, wherein the cumulative incidence of death, non-fatal myocardial infarction, stroke, and resuscitation is related to the initial concentration of PlGF in plasma (n=230). The patients were divided into groups according to the median PlGF concentrations of PlGF (17.7 ng/l).

FIG. 4 shows event-rates, calculated according to Kaplan-Meier, wherein the cumulative incidence of death, non-fatal myocardial infarction, stroke, and resuscitation is related to the initial concentrations of sFlt-1 in plasma (n=230). The patients were divided into groups according to the median sFlt-1 concentrations (56.5 ng/l).

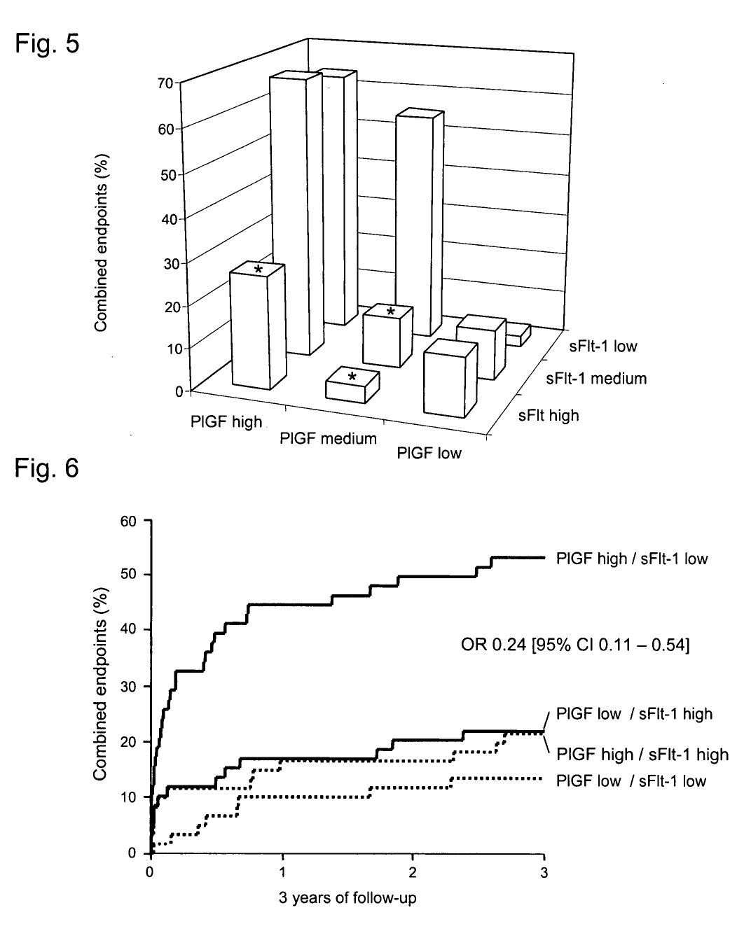

FIG. 5 shows the prognostic relevance of PlGF for the incidence of death, non-fatal myocardial infarction, stroke, and resuscitation related to the sFlt-1-concentrations. The patients were divided into tertiles according to the PlGF-concentrations (<15.6; 15.6-23.3; >23.3 ng/l) and to the sFlt-1 concentrations (<37.4; 37.4-91.4; >91.4 ng/l) (n=230), respectively.

FIG. 6 shows event-rates, calculated according to Kaplan-Meier, wherein the cumulative incidence of death, non-fatal myocardial infarction, stroke, and resuscitation is related to the initial concentrations of Flt-1 and PlGF (n=230), respectively. The patients were divided into groups according to the median concentrations of sFlt-1 and PlGF.

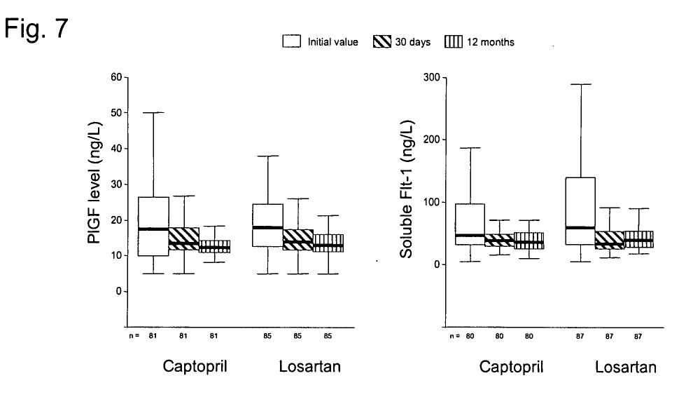

FIG. 7 shows changes in the concentrations of PlGF and sFlt-1, respectively, related to a randomised treatment during the further observation. The samples were collected at the beginning (initial value), after 30 days, and after 12 months (n≧80).

### A. PATIENTS AND METHODS

### 1. Patients

The patients who were examined were those who were already involved in the OPTIMAAL study (optimal trial in myocardial infarction with angiotensin II antagonist losartan) and who had experienced a myocardial infarction. The design and the most important results of the OPTIMAAL study were already described earlier (11). The study comprised a group of 230 patients diagnosed with myocardial infarction and a dysfunction of the left ventricle and/or a heart failure during the acute phase of the myocardial infarction. The patients were randomly divided into groups and adjusted to a dosage of losartan (1×50 mg/day) or captopril (3×50 mg/day), in accordance with compatibility. There were no substantial differences between both groups as treated regarding the initial characteristics.

### 2. Biochemical Analysis

Blood was drawn from the patients in the morning in a fasted state, wherein the blood samples were collected in pyrogen-free vacuum tubes with EDTA. The tubes were immediately immersed in ice-water, centrifuged within 15 minutes (1,000 g, 4° C., 15 minutes), and the plasma was stored as a multitude of aliquots at −80° C. until analysis. The determination of the markers were performed blinded, i.e., without knowledge of the patients' histories and treatment as assigned, in the central laboratory of the University of Frankfurt. PlGF, VEGF, sFlt-1, and sCD40 ligand (sCD40L) were measured using the ELISA technique (all reagents from R&D Systems, Wiesbaden) (7, 12, 13). Highly sensitive C-reactive protein (hsCRP) was measured using the Behring BN II Nephelometer (Dade-Behring, Deerfield, Ill.) (14).

### 3. Endpoints of Study

In connection with the study, an end point was determined which was composed of several parameters. The end point included overall mortality independent from the cause of death, resuscitation after cardiac arrest, re-occurring of non-fatal myocardial infarction, and stroke. A detailed description of the design and organization of the OPTIMAAL study has already been published earlier (11, 15).

### 4. Statistic Methods

A logistic regression model was used in order to determine the relative risk for vascular events (16). The separation into groups took place on the basis of the median concentration of each biomarker. A logistic regression model was used in order to determine the relative risk of death, non-fatal myocardial infarction, stroke and the need for resuscitation (16). The effects of the initial characteristics and biochemical markers on each of the relationships between PlGF concentrations and sFlt-1 concentrations, respectively, and vascular events, as examined, were analyzed through the stepwise functioning logistic regression model. All results that were obtained for continuous variables are given as mean value±standard deviation. Comparisons between the groups were analyzed by the t-test (two-sided). A comparison of the categorical variables was made by the Pearson χ²-test. Values of p<0.05 were regarded as statistically significant. All analyses were performed using the software SPSS 11.5 (SPSS Inc., Chicago, Ill.).

Statistical parameters are: n=230, lacking 10; median_{(PlGF)}=17.7250, median_{(sFlt-1)}=56.5000; percentile=33.33333333, 15.5700, 37.4300, 66.66666667, 23.2700, 91.4100.

The analysis according to Kaplan-Meyer represents a statistic standard method for the calculation of differences in the rate of death or the rate of an event-free survival.

### B. RESULTS

The initial concentrations of sFlt-1 in plasma showed a mean value of 183.2±465.6 ng/l (range of 5.0 to 2503.4), and the initial concentrations of PlGF in plasma were 24.0±20.0 ng/l (range of 5.0 to 144.9). When the sFlt-1-plasma concentrations were correlated to traditional biomarkers, no correlation with hsCRP concentrations (rank correlation coefficient according to Spearman r=0.12; p=0.08) was found, whereas the bi-variable correlation analysis showed a significant inverse correlation between sFlt-1 and sCD40L, although the correlation coefficients of r=0.17 (p=0.018) were low. In addition, no significant correlation between VEGF (r=0.03; p=0.66) or PlGF (r=0.05; p=0.44) and sFlt-1 plasma concentrations (FIG. 1), respectively, was found, although the sFlt-1-concentrations were significantly higher in patients with elevated PlGF-concentrations (FIG. 2).

### EXAMPLE 1

### Relationship Between Vascular Events and the Plasma Concentrations of PlGF and sFlt-1

The patients were divided according to their median concentrations of biomarkers. The initial characteristics differed in patients with high PlGF concentrations and patients with low PlGF concentrations only with respect to the sFlt-1-concentrations (Table 1). In patients with elevated PlGF concentrations, the event-rates for the combined end points of mortality, non-fatal. myocardial infarction, stroke, and reuscitation resuscitation were significantly higher (38.8% vs. 18.3%; p=0.001) (FIG. 3) compared to those with low PlGF concentrations. With reference to the most important vascular events (death and non-fatal myocardial infarction), the differences persisted with an event rate of 30.4% in patients with elevated PlGF concentrations, compared to 15.7% in patients with low PlGF-concentrations (odds ratio 2.36 [95% CI 1.24-4.48]; p=0.012).

The initial characteristics differed in patients with high sFlt-1 concentrations and patients with low sFlt-1-concentrations in view of the concentrations of BNP, sCD40L, and PlGF, and the incidence of new Q-waves in the ECG and the duration of hospitalization (Table 1). In patients with elevated sFlt-1 concentrations the event-rates for the combined end points of mortality, non-fatal myocardial infarction, stroke, and resuscitation tended to be lower than in patients with low sFlt-1 concentrations (22.6% vs. 33.9%; p=0.08) (FIG. 4). A non-significant difference was observed for the most important vascular events (death and non-fatal myocardial infarction) in 19.1% of the patients with elevated sFlt-1-concentrations compared to 27.0% in patients with low sFlt-1-concentrations (odds ratio 0.64 [95% CI 0.34-1.19]; p=0.21).

TABLE 1
Basic characteristics with respect to the plasma concentrations of PlGF and sFlt-1

| | I. PLGF | II. PLGF | III. sFlt-1 | IV. sFlt-1 |
|---|---|---|---|---|
| | low | high | low | high |
| N | 115 | 115 | 115 | 115 |
| Male | 65.2% | 75.7% | 66.1% | 74.8% |
| Age (years) | 66.6 ± 10.3 | 69.0 ± 10.4 | 68.7 ± 10.1 | 66.9 ± 10.6 |
| Newly occurring | 73.6% | 76.6% | 67.3% | 83.2% * |
| Q-waves | | | | |
| Anterior-wall infarction | 60.0% | 61.7% | 58.3% | 63.5% |
| Classification according | I: 20.0%; | I: 19.1%; | I: 15.7%; | I: 23.5%; |
| to Killip | II 65.2%; | II 65.2%; | II 73.0%; | II 57.4%; |
| | III 13.9%; | III 11.3%; | III 9.6%; | III 15.7%; |
| | IV 0.9% | IV 4.3% | IV 1.7% | IV 3.5% |
| Hospitalization (days) | 12.1 ± 20.1 | 14.2 ± 26.4 | 17.2 ± 28.0 | 9.1 ± 17.0 * |

History of patient

| | | | | |
|---|---|---|---|---|
| Angina | 19.1% | 25.2% | 27.0% | 17.4% |
| Myocardial infarction | 13.9% | 9.6% | 13.0% | 10.4% |
| PTCA | 3.5% | 0 | 1.7% | 1.7% |
| CABG | 1.7% | 0.9% | 1.7% | 0.9% |
| Diabetes | 11.3% | 11.3% | 11.3% | 11.3% |
| Hypertension | 32.2% | 31.3% | 29.6% | 33.9% |
| Active smoker | 35.7% | 43.5% | 39.1% | 40.0% |

Medication

| | | | | |
|---|---|---|---|---|
| Aspirin | 94.8% | 97.4% | 95.7% | 96.5% |
| Statins | 61.7% | 64.3% | 65.2% | 60.9% |
| Loop diuretics | 74.8% | 72.2% | 80.9% | 66.1% |
| Beta-blocker | 76.5% | 74.8% | 77.4% | 73.9% |
| BNP (pg/ml) | 125.2 ± 93.6 | 152.3 ± 126.4 | 115.5 ± 79.8 | 162.0 ± 132.9 * |
| CRP (μg/ml) | 66.7 ± 66.5 | 74.0 ± 64.1 | 75.2 ± 69.7 | 65.5 ± 60.5 |
| sCD40L (pg/ml) | 4228 ± 3943 | 3915 ± 4376 | 4906 ± 4340 | 3237 ± 3809 * |
| sFlt-1 (pg/ml) | 108.8 ± 268 | 257.7 ± 593.5 * | n.a. | n.a. |
| PlGF (pg/ml) | n.a. | n.a. | 18.5 ± 15.1 | 29.5 ± 22.7 * |

### EXAMPLE 2

### Interaction Between PlGF and sFlt-1

Patients with elevated PlGF concentrations also showed elevated concentrations of sFlt-1 (FIG. 2). Nevertheless, the sFlt-1 concentrations of both groups overlapped in a substantial range indicating that, surprisingly, the compensatory increase of the sFlt-1 concentrations in patients with elevated PlGF concentrations is inconsistent and can not be observed in all patients. Patients with PlGF concentrations in the two upper tertiles who, nevertheless, did not show an increase in the sFlt-1 concentrations (lower tertile), showed adverse after-effects compared to patients who exhibited sFlt concentrations in the uppermost tertile, but similarly elevated PlGF concentrations (FIG. 5). When the PlGF concentrations were only slightly elevated (second tertile), even a moderate increase in the sFlt-1 concentrations appeared to protect the patients from adverse after-effects. In contrast, in patients with strongly elevated concentrations of PlGF (third tertile), only those patients with sFlt-1 concentrations in the uppermost tertile showed a significantly lower event-rate. When the patients were divided into two groups on the basis of their PlGF and sFlt-1 concentrations, respectively, the prognosis of the patients with high sFlt-1 concentrations did not differ significantly from those patients with either high or low PlGF concentrations (FIG. 6). Accordingly, the ratio of PlGF and sFlt-1 is a powerful independent parameter for a prediction of vascular events (odds ratio 4.00 [95% CI 2.14-7.23]; p<0.001), which is significantly superior to the exclusive determination of one of the parameters. The event-rates in patients with low PlGF-concentrations were 14.0% and were independent from the sFlt-1-concentrations (p=0.95). In contrast, the event-rates in patients with high PlGF-concentrations were 55.8%, if the sFlt-1-concentrations were low, but 24.3%, if the sFlt-1-concentrations were elevated (p=0.002).

In summary, FIG. 6 demonstrates that:
(a) A ratio of [PlGF=high:sFlt-1=low] indicates a high risk for the patient for an adverse event such as death, non-fatal myocardial infarction, and stroke.
(b) In contrast, if the PlGF value is low, the risk for an adverse event is markedly lowered, regardless of whether or not the sFlt-1-value is high or low.
(c) At a ratio of [PlGF=low:sFlt-1=low], the risk for an adverse event is particularly low.
(d) If the sFlt-1-value is high, the risk for an adverse event is markedly lowered, regardless of whether or not the PlGF-value is high or low.

### EXAMPLE 3

### Multivariable Regression Analysis

In order to further examine the potential prognostic independence of individual biomarkers, a stepwise multivariable logistic regression analysis was performed, comprising PlGF and sFlt-1, as well as further biochemical markers, such as BNP, a marker of neurohumoral activation, hsCRP, a classical acute phase protein, and sCD40L, a marker of thromboinflammatory activation. In addition, basic characteristics were taken into account that showed a significant prognostic meaning in an univariable model. For the combined end points after a four-year observation period, only two established risk factors, namely advanced age and diabetes, were found as independent prognostic parameters, after the biochemical markers were included in the model (Table 2). The markers BNP (p=0.043), sCD40L (p=0.007), PlGF (p=0.001), and sFlt-1 (p=0.006) remained important and independent prognostic parameters for the further disease progression, whereas hsCRP lost somewhat of importance after PlGF was introduced into the model (p=0.77 after introduction of PlGF).

TABLE 2
Multivariate logistic regressions model for myocardial
infarction with fatal and non-fatal outcome during
the course of a four-year follow-up period

| | | 95% confidence | |
|---|---|---|---|
| Variable | odds-ratio | interval | p-value |
| Age > 75 years | 2.49 | 1.13-5.47 | 0.023 |
| Diabetes mellitus | 3.06 | 1.13-8.29 | 0.028 |
| Hypercholesterolemia | 0.77 | 0.29-2.00 | 0.59 |
| BNP > 113 ng/l | 2.09 | 1.03-4.25 | 0.04 |
| C-reactive protein > 50.0 mg/l | 1.11 | 0.55-2.25 | 0.77 |
| sCD40L > 3.5 μg/l | 2.70 | 1.31-5.58 | 0.007 |
| PlGF > 17.7 ng/l | 5.07 | 2.35-10.02 | 0.001 |
| sFlt-1 > 56.5 ng/l | 0.35 | 0.16-0.73 | 0.006 |
| PlGF · sFlt-1 | 3.11 | 2.03-3.88 | 0.001 |

### EXAMPLE 4

### Changes of the Biomarkers During the Observation Period

In agreement with the results of the study, which were derived from the overall group of the patients, no difference in clinical progression was found between the captopril and the losartan treatment groups. In addition, neither in patients with high nor in patients with low PlGF concentrations was a reduction of the events observed (PlGF low: 19% event-rate in the captopril group vs. 17.5% in the losartan group; p=1.00; PlGF high: 41.1% vs. 35.6%; p=0.57). Similar results were obtained for the sFlt-1-concentrations: sFlt-1 high: 22.2% in the captopril group vs. 23.9% in the losartan group (p=1.00); sFlt-1 low: 36.7% in the captopril-group vs. 30.9% in the group vs. 30.9% in the losartan-group (p=0.56). It was furthermore found that in patients of whom serial samples were available (day 0, 30 days, and 1 year; n≧80 for each group and each time point) both the PlGF and the sFlt-1 concentrations continuously decreased during the observation period, no differences occurring between the treatment groups (FIG. 7).

The results of the study show that elevated blood levels of PlGF are connected to vascular events in patients after a myocardial infarction. In agreement with a new study on patients with acute coronary heart diseases (7), the prognostic importance of the concentrations of PlGF in plasma was independent from other biomarkers representing distinct pathophysiological processes. Elevated PlGF concentrations provided a prognostic value which had more significance than information derived from hsCRP plasma concentrations. Through multivariate regression analysis, several other biochemical markers, including B-type natriuretic peptide, a marker of neurohumoral activation, sCD40L, a marker of thrombo-inflammatory activation, and PlGF, a marker of vascular inflammation, were identified as independent prognostic parameters for the further progression of the disease during the following four years. Nevertheless, the new and most important finding of the study is that the prognostic importance of PlGF is modulated by sFlt-1. These findings show that the balance between PlGF and its soluble receptor sFlt-1 as the only known endogenous regulator is an essential determinant in view of the further disease progression in patients with acute myocardial infarction.

Both the reason of the elevated concentrations of sFlt-1 as well as the signals which up-regulate the Flt-1 expression in patients which have experienced an acute myocardial infarction currently are not known. Hypoxia is a potent stimulus for the up-regulation of the Flt-1-expression (6, 19). It is possible that a large portion of sFlt-1 is released from the inflammatory cells by so-called shedding (3, 9, 20). Independent of the mechanisms that are involved in the increase of the concentrations of sFlt-1 in plasma, the results of the present study emphasize the key role of the balance between pro- and anti-inflammatory mediators for the risk stratification in the context of an acute coronary heart disease (21).

In particular, these studies give rise to the hope that new anti-inflammatory strategies can be developed in order to counteract the progression of a manifested atherosclerosis. The infusion of sFlt-1 with the purpose to reduce the concentrations of circulating active PlGF in patients with unstable or rapidly progressing coronary heart disease could be particularly effective in those patients who have elevated PlGF concentrations and low concentrations of its inhibitor sFlt-1.

The results of the present study show that elevated plasma concentrations of PlGF, a marker of vascular inflammation, in patients after a myocardial infarction is correlated with an elevated risk for subsequent vascular events. Nevertheless, the informational value with regard to prognosis depends on the concentration of sFlt-1, which supports the hypothesis that sFlt-1 regulates the activity of PlGF through binding and inactivation. These findings could provide the basis of a new anti-inflammatory therapeutic approach using sFlt-1 in order to reduce circulating PlGF in patients who have an elevated risk for an adverse vascular event.

### LIST OF REFERENCES

Braunwald E. Unstable angina: an etiologic approach to management. Circulation. 1998; 98:2219-22.
Libby P, Ridker P M, Maseri A. Inflammation and atherosclerosis. Circulation. 2002; 105:1135-43.
Luttun A, Tjwa M, Moons L, Wu Y, Angelillo-Scherrer A, Liao F, Nagy J A, Hooper A, Priller J, De Klerck B, Compernolle V, Daci E, Bohlen P, Dewerchin M, Herbert J M, Fava R, Matthys P, Carmeliet G, Collen D, Dvorak H F, Hicklin D J, Carmeliet P. Revascularization of ischemic tissues by PlGF treatment, and inhibition of tumor angiogenesis, arthritis and atherosclerosis by anti-Flt1. Nat Med. 2002;8:831-40.
Maglione D, Guerriero V, Viglietto G, Delli-Bovi P, Persico M G. Isolation of a human placenta cDNA coding for a protein related to the vascular permeability factor. Proc Natl Acad Sci USA. 1991;88:9267-71.
Autiero M, Luttun A, Tjwa M, Carmeliet P. Placental growth factor and its receptor, vascular endothelial growth factor receptor-1: novel targets for stimulation of ischemic tissue revascularization and inhibition of angiogenic and inflammatory disorders. J Thromb Haemost. 2003;1:1356-70.
Luttun A, Tjwa M, Carmeliet P. Placental Growth Factor (PlGF) and Its Receptor Flt-1 (VEGFR-1): Novel Therapeutic Targets for Angiogenic Disorders. Ann N Y Acad Sci. 2002;979:80-93.
Heeschen C, Dimmeler S, Fichtlscherer S, Hamm C W, Berger J, Simoons M L, Zeiher A M. Prognostic value of placental growth factor in patients with acute chest pain. Jama. 2004;291 :435-41.
Khaliq A, Dunk C, Jiang J, Shams M, Li X F, Acevedo C, Weich H, Whittle M, Ahmed A. Hypoxia down-regulates placenta growth factor, whereas fetal growth restriction up-regulates placenta growth factor expression: molecular evidence for “placental hyperoxia” in intrauterine growth restriction. Lab Invest. 1999;79:151-70.
Rafii S, Avecilla S, Shmelkov S, Shido K, Tejada R, Moore M A, Heissig B, Hattori K. Angiogenic factors reconstitute hematopoiesis by recruiting stem cells from bone marrow microenvironment. Ann N Y Acad Sci. 2003;996:49-60.
Chung N A, Makin A J, Lip G Y. Measurement of the soluble angiopoietin receptor tie-2 in patients with coronary artery disease: development and application of an immunoassay. Eur J Clin Invest. 2003;33:529-35.
Dickstein K, Kjekshus J. Effects of losartan and captopril on mortality and morbidity in high-risk patients after acute myocardial infarction: the OPTIMAAL randomised randomized trial. Optimal Trial in Myocardial Infarction with Angiotensin II Antagonist Losartan. Lancet. 2002;360:752-760.
Heeschen C, Dimmeler S, Hamm C W, van den Brand M J, Boersma E, Zeiher A M, Simoons M L. Soluble CD40 ligand in acute coronary syndromes. N Engl J Med. 2003;348:1104-11.
Heeschen C, Dimmeler S, Hamm C W, Boersma E, Zeiher A M, Simoons M L. Prognostic significance of angiogenic growth factor serum levels in patients with acute coronary syndromes. Circulation. 2003;107:524-530.
Heeschen C, Hamm C W, Bruemmer J, Simoons M L. Predictive value of C-reactive protein and troponin T in patients with unstable angina: a comparative analysis. CAPTURE Investigators. Chimeric c7E3 AntiPlatelet Therapy in Unstable angina REfractory to standard treatment trial. J Am Coll Cardiol. 2000;35:1535-42.
Dickstein K, Kjekshus J. Comparison of the effects of losartan and captopril on mortality in patients after acute myocardial infarction: the OPTIMAAL trial design. Optimal Therapy in Myocardial Infarction with the Angiotensin II Antagonist Losartan. Am J Cardiol. 1999;83:477-81.
Cox D R. Regression models and life-tables. J R Stat Soc [B]. 1972;34:187-220.
Maynard S E, Jiang-Yong Min J-Y, Merchan J, Lim K H, Li J, Mondal S, Libermann T A, Morgan J P, Sellke F W, Stillman I E, Epstein F H, Sukhatme V P, Karumanchi S A. Excess placental soluble fms-like tyrosine kinase 1 (sFlt1) may contribute to endothelial dysfunction, hypertension, and proteinuria in preeclampsia. J. Clin. Invest. 2003;1 11:649-658.
Levine R J, Maynard S E, Qian C, Lim K H, England L J, Lu K F, Schisterman E F, Thadhani R, Sachs B P, Epstein F H, Sibai B M, Sukhatme V P, Karumanchi S A. Circulating Angiogenic Factors and the Risk of Peeelampsioa Preeclampsioa. N Engl J Med. 2004;350:672-683.
Takeda N, Maemura K, Imai Y, Harada T, Kawanami D, Nojiri T, Manabe I, Nagai R. Endothelial PAS domain protein 1 gene promotes angiogenesis through the transactivation of both vascular endothelial growth factor and its receptor, Flt-1. Circ Res. 2004;95:146-53.
Selvaraj S K, Giri R K, Perelman N, Johnson C, Malik P, Kalra V K. Mechanism of monocyte activation and expression of proinflammatory cytochemokines by placenta growth factor. Blood. 2003;102:1515-24.
Heeschen C, Dimmeler S, Hamm C W, Fichtlscherer S, Boersma E, Simoons M L, Zeiher A M. Serum level of the antiinflammatory cytokine interleukin-10 is an important prognostic determinant in patients with acute coronary syndromes. Circulation. 2003;107:2109-14.
Ridker P M. Clinical Application of C-Reactive Protein for Cardiovascular Disease Detection and Prevention. Circulation 2003; 107:363-369.
Koenig W, Lowel H, Baumert J, Meisinger C. C-reactive protein modulates risk prediction based on the Framingham Score: implications for future risk assessment: results from a large cohort study in southern Germany. Circulation 2004; 109:1349-53.
Danesh J, Wheeler J G, Hirschfield G M, et al. C-reactive protein and other circulating markers of inflammation in the prediction of coronary heart disease. N Engl J Med 2004; 350:1387-97.
Morrow D A, Rifai N, Antman E M, et al. C-reactive protein is a potent predictor of mortality independently of and in combination with troponin T in acute coronary syndromes: a TIMI 11A substudy. Thrombolysis in Myocardial Infarction. J Am Coll Cardiol 1998; 31:1460-5.
Lindahl B, Toss H, Siegbahn A, Venge P, Wallentin L. Markers of myocardial damage and inflammation in relation to long-term mortality in unstable coronary artery disease. FRISC Study Group. Fragmin during Instability in Coronary Artery Disease. N Engl J Med 2000; 343:1139-47.
James S K, Lindahl B, Siegbahn A, et al. N-terminal pro-brain natriuretic peptide and other risk markers for the separate prediction of mortality and subsequent myocardial infarction in patients with unstable coronary artery disease: a Global Utilization of Strategies To Open occluded arteries (GUSTO)-IV substudy. Circulation 2003; 108:275-81.

1-33. (canceled)

34. An in vitro method of diagnosing, stratifying the risk of, monitoring, and/or estimating the probability of developing a vascular disease of atherosclerotic etiology, comprising:
(a) providing a patient sample for analysis;
(b) quantifying the PlGF in the sample; and
(c) quantifying the sFlt-1 in the sample,
wherein the levels of PlGF and sFlt-1 correlate with the presence of a vascular disease of atherosclerotic etiology.

35. The method of claim 34, further comprising at least one of:
(d) comparing the quantity of PlGF obtained in (b) to a reference sample of PlGF, and comparing the quantity of sFlt-1 obtained in (c) to a reference sample of FLT-1;
(e) determining the ratio of the quantity of PlGF obtained in (b) and the quantity of sFlt-1 obtained in (c); and
(f) comparing the ratio obtained in (e) to the ratio of PlGF to sFlt-1 in a reference sample.

36. The method of claim 34, wherein the vascular disease is selected from coronary heart disease, cerebrovascular disease, and peripheral arterial occlusive disease.

37. The method of claim 36, wherein the coronary heart disease is an acute coronary syndrome.

38. The method of claim 37, wherein the acute coronary syndrome is unstable angina pectoris and/or acute myocardial infarction.

39. The method of claim 34, wherein the sample for analysis is peripheral blood or a fraction thereof.

40. The method of claim 39, wherein the peripheral blood fraction is either serum or plasma.

41. The method of claim 34, wherein the risk stratification comprises determining a probability of an adverse event selected from death, non-fatal myocardial infarction, and stroke.

42. The method of claim 41, wherein a quantity of PlGF above a reference value of ≧ about 15.6 ng/l PlGF and a quantity of sFlt-1 below a reference value of less than or equal to about 56.5 ng/l sFlt-1 indicates an elevated probability of the adverse event.

43. The method of claim 42, wherein the quantity of PlGF is above a reference value of greater than or equal to about 17.7 ng/l and the quantity of sFlt-1 is below a reference value of less than or equal to about 56.5 ng/l.

44. The method of claim 43, wherein the quantity of PlGF is above a reference value of greater than or equal to about 23.3 ng/l and the quantity of sFlt-1 is below a reference value of less than or equal to about 56.5 ng/l.

45. The method of claim 42, wherein the quantity of PlGF is above a reference value of greater than or equal to about 15.6 ng/l and the quantity of sFlt-1 is below a reference value of less than or equal to about 37.4 ng/l.

46. The method of claim 45, wherein the quantity of PlGF is above a reference value of greater than or equal to about 17.7 ng/l and the quantity of sFlt-1 is below a reference value of less than or equal to about 37.4 ng/l.

47. The method of claim 46, wherein the quantity of PlGF is above a reference value of greater than or equal to about 23.3 ng/l and the quantity of sFlt-1 is below a reference value of less than or equal to about 37.4 ng/l.

48. The method of claim 41, wherein a PlGF concentration in the upper two tertiles of a reference collective, and an sFlt-1 concentration in the lower tertile of the reference collective, indicates an elevated probability of the adverse event.

49. The method of claim 41, wherein a ratio of [PlGF: sFlt-1] of greater than or equal to about 0.31 ng/l indicates an elevated probability of the adverse event.

50. The method of claim 49, wherein a ratio of [PlGF: sFlt-1] of greater than or equal to about 0.42 ng/l indicates an elevated probability for an adverse event.

51. The method of claim 50, wherein a ratio of [PlGF: sFlt-1] of greater than or equal to about 0.62 ng/l indicates an elevated probability for an adverse event.

52. The method of claim 34, further comprising quantifying at least one biomarker selected from VEGF, sCD40L, PAPP-A, MPO, myoglobin, creatine kinase, troponin, CRP, cystatin C, and natriuretic peptides.

53. The method of claim 52, wherein the creatine kinase is CK-MB.

54. The method of claim 52, wherein the troponin is selected from troponin I, troponin T, and a complex of either troponin I or troponin T.

55. The method of claim 52, wherein the natriuretic peptide is selected from ANB, BNP, and/or NT-proBNP.

56. The method of claim 34, wherein the patient is treated with one or more therapeutic agents selected from sFlt-1, an anti-inflammatory agent, an anti-thrombotic agent, an anti-platelet agent, a fibrinolytic agent, a lipid lowering agent, a direct thrombin inhibitor, and a glycoprotein IIb/IIIa receptor inhibitor.

57. An in vitro method of identifying a patient for treatment, comprising:
(a) providing a patient sample for analysis;
(b) quantifying the PlGF in the sample; and
(c) quantifying the sFlt-1 in the sample,
wherein the patient may therapeutically benefit from one or more agent selected from sFlt-1, an anti-inflammatory agent, an anti-thrombotic agent, an anti-platelet agent, a fibrinolytic agent, a lipid lowering agent, a direct thrombin inhibitor, and a glycoprotein IIb/IIIa receptor inhibitor.

58. A diagnostic kit comprising:
(a) at least one means for quantifying PlGF in a sample to be analyzed;
(b) at least one means for quantifying sFlt-1 in a sample to be analyzed;
(c) at least one reference sample having a concentration of PlGF of greater than or equal to about 15.6 ng/l and/or a concentration of sFlt-1 of less than or equal to about 56.5 ng/l

59. The kit of claim 58, consisting of separate packaging units.

60. The kit of claim 58, wherein the reference sample has a PlGF concentration of greater than or equal to about 17.7 ng/l.

61. The kit of claim 60, wherein the reference sample has a PlGF concentration of greater than or equal to about 23.3 ng/l.

62. The kit of claim 61, wherein the reference sample has a sFlt-1 concentration of less than or equal to about 37.4 ng/l.

63. A method of using the kit of claim 58 comprising:
(a) providing a patient sample for analysis;
(b) quantifying the PlGF in the sample; and
(c) quantifying the sFlt-1 in the sample,
wherein the method provides for the diagnosis, risk stratification, estimation of the probability of developing, and/or monitoring of a vascular disease of atherosclerotic etiology.

64. A method of measuring PlGF and sFlt-1, using an assay element, comprising:
(a) providing a patient sample for analysis;
(b) quantifying the PlGF in the sample; and
(c) quantifying the sFlt-1 in the sample,
wherein the assay element comprises a sample application zone for application of the sample and for application of labeled specific PlGF binding partners and/or specific sFlt-1 binding partners,
wherein the sample application zone is contacting at least one detection zone,
wherein the detection zone comprises spatially separated regions for specific binding of PlGF and sFlt-1,
wherein the measurement provides for diagnosis, risk stratification, estimation of the probability of developing, and/or monitoring of a vascular disease of atherosclerotic etiology, and
wherein the measurement provides for identification of a patient for treatment with one or more therapeutic agents selected from sFlt-1, an anti-inflammatory agent, an anti-thrombotic agent, an anti-platelet agent, a fibrinolytic agent, a lipid lowering agent, a direct thrombin inhibitor, and a glycoprotein IIb/IIIa receptor inhibitor.

65. The method of claim 64, wherein the binding partners are present in the assay element.

66. The method of claim 64, wherein the assay element is an immunochromatic assay element.
